(19) United States
(12) Patent Application Publication
(10) Pub. No.: US 2021/0204900 A1
(43) Pub. Date: Jul. 8, 2021

(54) TOMOGRAPHY APPARATUS

(71) Applicant: National University Corporation Chiba University, Chiba (JP)

(72) Inventor: Hideyuki KAWAI, Chiba (JP)

(21) Appl. No.: 16/650,417

(22) PCT Filed: Aug. 16, 2018
(86) PCT No.: PCT/JP2018/030420

§ 371 Date: Mar. 25, 2020

(30) Foreign Application Priority Data
Oct. 20, 2017 (JP) 2017-203860

(51) Int. Cl. A61B 6/00; G01T 1/20; A61B 6/03

(52) U.S. Cl. CPC A61B 6/542; A61B 6/03; G01T 1/20185

(57) ABSTRACT

The exposure dose of a subject by X-ray CT examination is reduced. In order to do so, a tomography apparatus has been made to include: a γ-ray generation source configured to irradiate a measurement target with γ-rays; a measurement unit configured to measure information relating to transmittance of a γ-ray that has passed through the measurement target; and a substance density distribution calculation unit configured to calculate a substance density distribution of the measurement target based on the information relating to the transmittance of the γ-ray measured by the measurement unit. In another embodiment, it is desirable to use a detector that combines γ-rays with a scintillator and optical fibers.

### BACKGROUND

### 1. Technical Field

The present invention relates to a tomography apparatus (computerized tomography; CT).

### 2. Related Art

X-ray CT is an indispensable diagnostic method in current medical care in Japan. In addition, it is useful as a nondestructive inspection method for industrial use. It is noted that in the present description, a tomography apparatus may be expressed as a tomographic measurement apparatus.

Non-Patent Document 1 describes an X-ray CT that irradiates a sample with X-rays and measures the density, shape, and the like of the sample from the attenuation of the X-rays transmitted through the sample.
Non-Patent Document 1: https://www.toyo.co.jp/microscopy/products/list/?contents_type=48

### GENERAL DISCLOSURE

Japanese people are exposed to 3 mSv per year for medical treatment. It is estimated that approximately 4% of all cancer patients developed cancer due to this practice, which is 4 to 6 times that of Europe and the United States. Most of the medical exposure is likely to be due to X-ray CT. Simple X-ray photography has an exposure of about 70 μSv. In X-ray CT, a radioactive ray of about 20 mSv occurs because a three-dimensional substance distribution is obtained from about 300 two-dimensional fluoroscopic images photographed from various directions. In this way, when X-ray CT is used for diagnostic purposes, health damage due to exposure becomes an issue.

In addition, X-ray CT is also used industrially for nondestructive internal fluoroscopy of metal products with complex structures. The higher the energy of the X-rays, the higher the transmittance and thickness of the member that can be seen through, but the higher the energy, the more complex and expensive the X-ray generation apparatus, and generally the highest energy of X-rays that can be artificially generated is about 200 keV.

When a substance having a low atomic number such as an organic substance is present in a container made of a substance having a high atomic number such as a metal, it is difficult in principle to see through with X-rays.

Therefore, an object of the present invention is to provide a tomography apparatus (tomographic measurement apparatus) that can reduce the exposure dose of a person to be diagnosed when the tomography apparatus is used for diagnostic purposes.

In addition, another object of the present invention is to provide a tomography apparatus capable of seeing through a substance having a high atomic number.

In order to solve the above problems, according to one aspect of the present invention, a tomographic apparatus includes a γ-ray generation source configured to irradiate a measurement target with γ-rays; a measurement unit configured to measure information relating to transmittance of a γ-ray that has passed through the measurement target; and a substance density distribution calculation unit configured to calculate a substance density distribution of the measurement target based on the information relating to the transmittance of the γ-ray measured by the measurement unit.

In addition, according to another aspect of the present invention, a tomography apparatus includes a γ-ray generation source configured to emit a first γ-ray in a direction of a measurement target and emit a second γ-ray to an opposite side of the measurement target; a first scintillator configured to receive the first γ-ray emitted from the γ-ray generation source to emit light; a plurality of first optical fibers disposed on a surface of the first scintillator and configured to re-emit light when light from the first scintillator is made incident thereon; a first light-receiving element attached to at least one end of each of the first optical fibers and configured to measure a physical quantity relating to the light re-emitted by the first optical fibers; a second scintillator configured to receive the second γ-ray emitted from the γ-ray generation source to emit light; a plurality of second optical fibers disposed on a surface of the second scintillator and configured to re-emit light when the light from the second scintillator is made incident thereon; a second light-receiving element attached to at least one end of each of the second optical fibers and configured to measure a physical quantity relating to the light re-emitted by the second optical fibers; and a substance density distribution calculation unit configured to calculate a substance density distribution of the measurement target based on the physical quantity relating to the light measured by the first light-receiving element and the physical quantity relating to the light measured by the second light-receiving element. Furthermore, it is desirable that a side surface of the first scintillator includes a first measurement element for measuring information relating to energy of the first γ-ray made incident on the first scintillator, a side surface of the second scintillator includes a second measurement element for measuring information relating to energy of the second γ-ray made incident on the second scintillator, and the substance density distribution calculation unit calculates the substance density distribution of the measurement target based on the physical quantity relating to the light measured by the first light-receiving element, the physical quantity relating to the light measured by the second light-receiving element, the information relating to the energy of the first γ-ray measured by the first measurement element, and the information relating to the energy of the second γ-ray measured by the second measurement element. Furthermore, it is desirable that the measurement target is a living body.

According to the present invention, it is possible to provide a tomography apparatus for medical diagnosis that can reduce the exposure dose of a person to be diagnosed. In addition, according to the present invention, it is possible to provide a tomography apparatus capable of seeing through a substance having a high atomic number.

### BRIEF DESCRIPTION OF THE DRAWINGS

FIG. 1 is a diagram showing a γ-ray measuring device according to a first working example.

FIG. 2 is a diagram showing a transmission-type γ-ray CT according to the first working example.

FIG. 3 is a diagram showing a scattering-type γ-ray CT of a second working example.

FIG. 4 is a diagram showing a schematic view of a CT according to a fifth working example.

FIG. 5 is a diagram showing a schematic view of a CT according to a sixth working example.

### DESCRIPTION OF EXEMPLARY EMBODIMENTS

Hereinafter, example embodiments and working examples of the present invention will be described, but the embodiments of the present invention are not limited to the example embodiments and the working examples described below.

The present invention is a γ-ray CT in which a reconstructed image equal to or better than that of an X-ray CT can be obtained and the exposure dose is lower than that of the X-ray CT. A ⁶⁸Ge/⁶⁸Ga sealed source is used for a γ-ray generation source.

### First Working Example

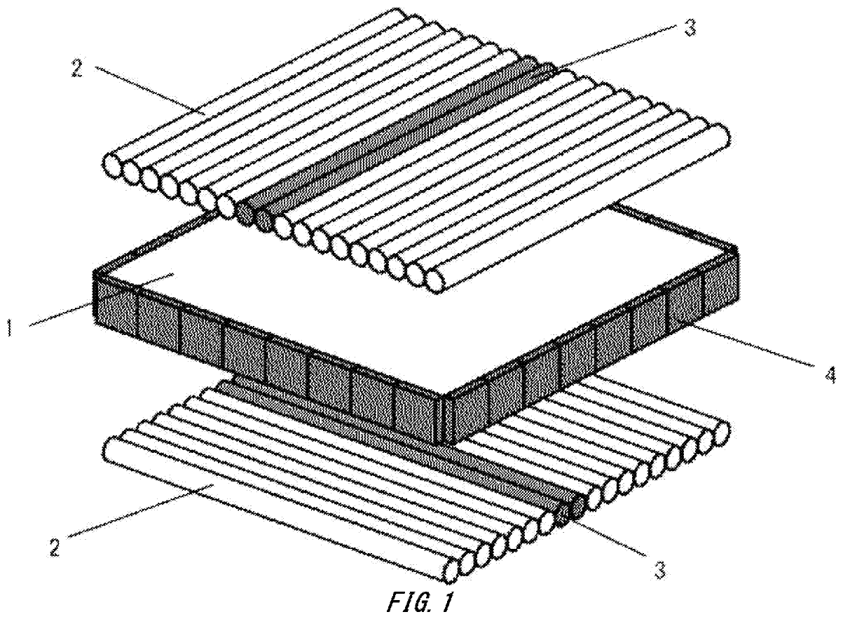

FIG. 1 is an example of a γ-ray measuring device proposed by the inventor. 1 denotes a La-GPS scintillator (GaₓLa_{1-x-y}Cey)2Si₂O₇ which emits light when γ-rays are made incident thereon, and has a size of 34 mm×34 mm×3.4 mm. The scintillator means a substance having a property of emitting light upon incidence of a radioactive ray, and as long as having this function, it is not limited to the material exemplified in the present working example. The length of each side exemplified here is a size of the maximum square that can be cut out from a cylindrical crystal having a diameter of 2 inches, and the thickness is adjusted to the outer shape of a small light-receiving element SiPM (product name of Hamamatsu Photonics, Inc.: Multi Pixel Photon Counter) having an effective area of 3 mm×3 mm.

Eight to ten MPPCs are adhered to each of the four sides of the scintillator plate, and the luminescence amount and luminescence time are measured. MPPC (Multi-Pixel Photon Counter) is a new type of photon counting (photon measuring) device (light-receiving element) that is a kind of device called a SiPM (Silicon Photomultiplier) and is a multi-pixel Geiger mode APD. In preliminary experiments to date, about 5,000 photoelectrons have been observed for 511 keV γ-rays. This corresponds to energy resolution of about 2%. In addition, time resolution is expected to be about 100 psec.

Two layers of 340 wavelength conversion fibers (optical fibers) 2 each having a diameter of 0.2 mm, are respectively adhered to the upper and lower surfaces of the scintillator plate. In the present diagram, the scintillation light is incident on wavelength conversion fibers 3, but the scintillation light incident on the core portions of the wavelength conversion fibers 3 is absorbed and isotropically re-emitted with a probability of about 50% as light having a slightly longer wavelength. About 10% of the light that satisfies the condition of total reflection in the fiber propagates to the end of the fiber and is observed by the MPPC adhered to the end. In preliminary experiments, 90 photoelectrons were observed at both ends for 511 keV γ-rays. This means that even with a scintillator plate that consumed only 50 keV in Compton scattering, the emission position can be measured with a probability of 99%. Since the measurement accuracy of the γ-ray incident position is determined by the diameter of the wavelength conversion fiber, a position resolution of up to 0.2 mm can be obtained.

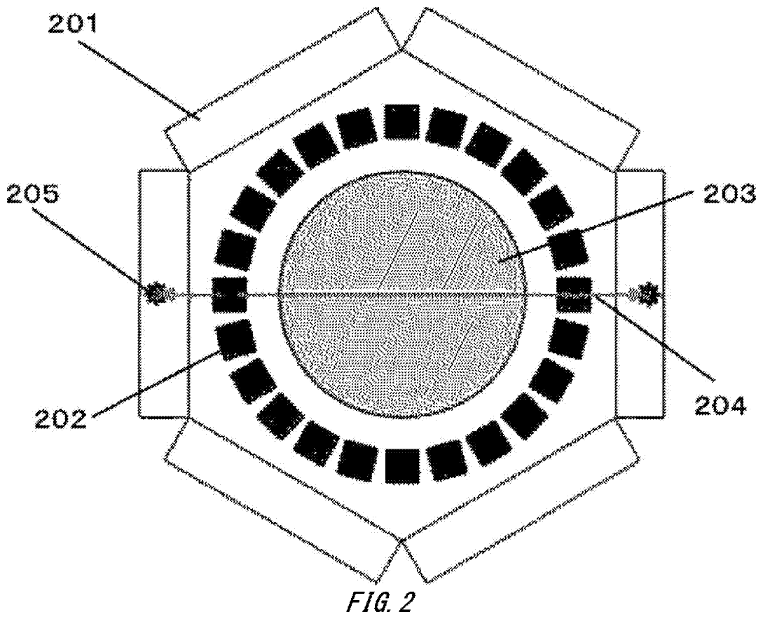

FIG. 2 is a diagram showing a transmission-type γ-ray CT. 203 denotes a living body to be measured, 202 denotes a sealed radiation source of a positron emitting nuclide, and 204 denotes a radioactive ray (γ-ray) emitted from the radiation source. The γ-ray is made incident on a scintillator 201, and is annihilated at 205, and scintillation light is emitted. It is noted that although a large number of radioactive rays are emitted isotropically and equally from the radiation source 202, the radioactive ray 204 will be described in the present working example. On the front surface and back surface of the scintillator, wavelength conversion fibers (not shown) are disposed side by side in two directions substantially orthogonal to each other, and the scintillation light is made incident on the wavelength conversion fibers. The γ-ray annihilation position is measured using the plate-like scintillator 201 and the wavelength conversion fibers. By measuring the γ-ray emitted on the opposite side of the living body, the expected position of the γ-ray transmitted through the living body is determined. A measurement is made of whether or not the γ-ray appears near the expected position. By analyzing this result, the three-dimensional density distribution and mass distribution of the measurement target are measured.

If the transmittance is determined with 1% accuracy with 70 keV X-rays, 2.22 million photons per unit area must be made incident so that the number of transmitted X-ray photons is 10,000 when it is assumed that there is no background due to Compton scattering. In this case, since the background (photons that arrived at the measuring device after Compton scattering) is 2.3 times the signal (photons that arrived at the measuring device without Compton scattering), to obtain the transmittance with the same accuracy, 24.2 million photons per unit area must be incident, where the number of events is 3.32 times that of the above case without the background. On the other hand, in the case of 511 keV γ-rays, since the expected arrival position of the transmitted photons is determined by the γ-ray measurement on the opposite side, it is only necessary to know from the transmittance measurement whether or not the photons are observed at the expected arrival position. The background is almost zero because events that it coincides with an expected arrival position after multiple Compton scatterings are negligible. In the case of 70 keV X-rays, the measurement efficiency is almost 100%, but in the case of 511 keV γ-rays, the measurement efficiency is about 90%. Therefore, to measure the transmittance with an accuracy of 1%, it is sufficient to measure 12,100 transmitted photons. Since the transmittance is 5.5%, 220,000 incident photons per unit area is sufficient. The initial photon energy is 7.3 times at 70 keV and 511 keV, and the ratio of photon energy consumed in the living body is 98.8% at 70 keV and 69.3% at 511 keV. Therefore, the exposure dose for the case of γ-rays is about 1/10 to 1/20 of the case of X-rays.

The accuracy of X-ray CT images is 1 to 2 mm. In the present working example, the exposure dose can be reduced to about 1/20 while improving the accuracy of the transmission image to ⅕ or less as compared with the conventional X-ray CT.

### Second Working Example

In addition, γ-ray CT can be also performed by the method proposed below.

Simple X-ray photography has an exposure of about 70 μSv. In X-ray CT, a radiation of about 20 mSv occurs because a three-dimensional substance distribution is obtained from about 300 two-dimensional fluoroscopic images photographed from various directions. In order to drastically reduce the exposure dose, instead of calculating the three-dimensional material distribution from two-dimensional fluoroscopic images as in X-ray CT, it can be considered to directly calculate the scattering point distribution=material distribution by measuring a radiation scattering phenomenon in the material. In Compton scattering, which is the scattering of a photon, the scattering angle is uniquely determined if the energy of the incident photon and scattered photon are known. The Compton camera determines the direction of the incident photon by measuring the position of the scattering point, the energy consumption, and the direction and energy of the scattered photon. Conversely, by measuring the position, direction, and energy of the incident photon and the position and energy of the scattered photon, the position of the scattering point can determined. In an ordinary X-ray generation apparatus, if a thick collimator is used, beam-like X-rays having a uniform traveling direction can be obtained, but it is impossible to know the energy of each X-ray photon. However, γ-rays generated by positron pair annihilation always have energy of 511 keV, and measuring the positron annihilation position and one γ-ray uniquely determines the incident position and traveling direction of the other γ-ray incident on the living body. If the position and energy of the scattered γ-ray are measured on the opposite side of the living body, the position where Compton scattering has occurred in the living body is uniquely determined.

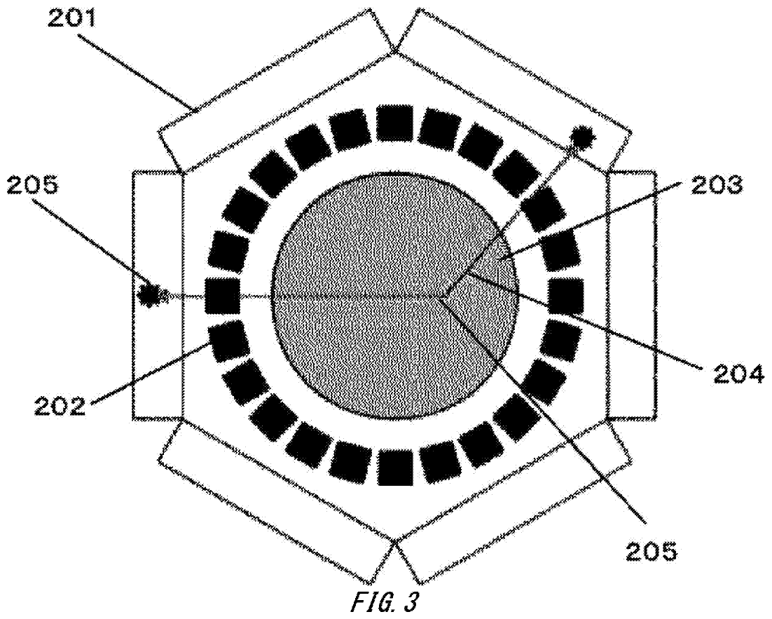

FIG. 3 is a diagram showing the concept of the scattering-type γ-ray CT of the present working example. If the position, direction, and energy of a photon made incident on the living body 203 and the position and energy of the photon exiting from the living body 203 can be measured, the scattering angle is uniquely determined, and the position 205 where the scattering occurs is determined for each event. From the information on the position where the scattering occurs, the substance density distribution in the living body is directly determined. Compared with the transmission-type γ-ray CT of the second working example, the number of events required for the substance distribution calculation is significantly reduced. In the present working example, the energy of the incident photon is self-evident, and the position and direction of incident light are determined from the measurement of the γ-ray on the opposite side of the positron annihilation position.

When Compton scattering events of 400 keV or more are used, the signal to noise ratio is 1:0.3, so to measure the material density in any 3 mm×3 mm×3 mm region with 1% accuracy, 16,900 of one-time Compton scattering events that scattered in the region should be measured. Since the ratio of a one-time Compton scattering event of 400 keV or more is 4.0%, the number of 511 keV photons incident on this region is 422,500, and the total energy is 3.45×10⁻⁸ J. Dividing this energy by the mass of this region, 2.7×10⁻⁵ kg, gives an exposure dose of 1.28 mSv in this region. This calculation assumes that all incident energy is consumed inside the body, but as mentioned in the above section, 30.7% of the energy is emitted outside the body by transmitted and scattered photons. In addition, in this γ-ray CT examination, the exposed site is a part of the whole body. Assuming that the ratio including the tissue weighting factor of the exposed site is 20%, the exposure dose of the whole body is 0.174 mSv. Then, according to the present working example, the exposure dose can be significantly reduced to about 1/100 as compared with the conventional X-ray CT. However, in the present working example, the position resolution is about 3 mm, which is slightly deteriorated as compared with X-ray CT.

### Third Working Example

If the first working example and the second working example are used together, a fluoroscopic examination with a low exposure dose and a high positional resolution can be realized. First, the whole body is seen through the scattering-type γ-ray CT of the second working example, and if an abnormal site is found, the transmission-type γ-ray CT of the first working example in which the γ-ray radiation range is limited to the vicinity of the abnormal site is used to perform a precise diagnosis.

As described above, the γ-ray CT diagnostic method of the present proposal using positron annihilation γ-rays from a positron-rich nucleus (for example, a ⁶⁸Ge/⁶⁸Ga sealed source) has the same ability to measure the substance distribution in a living body as X-ray CT. The diagnostic image accuracy and exposure dose compared to X-ray CT are respectively about ⅕ and 1/10 for the transmission-type γ-ray CT of the present proposal, about 2 times and 1/100 for the scattering-type γ-ray CT, and about ⅕ and 1/50 when both are used in combination.

### Fourth Working Example

In the above working example, the usage example of the medical diagnostic γ-ray CT has been described. However, since the γ-ray CT allows seeing through the inside of a substance having a high atomic number, it can also be used industrially, for luggage inspection in airports or the like, for fluoroscopic inspection of passenger cars, trucks, containers, etc., and for safety inspection of reinforced concrete structures.

### Fifth Working Example

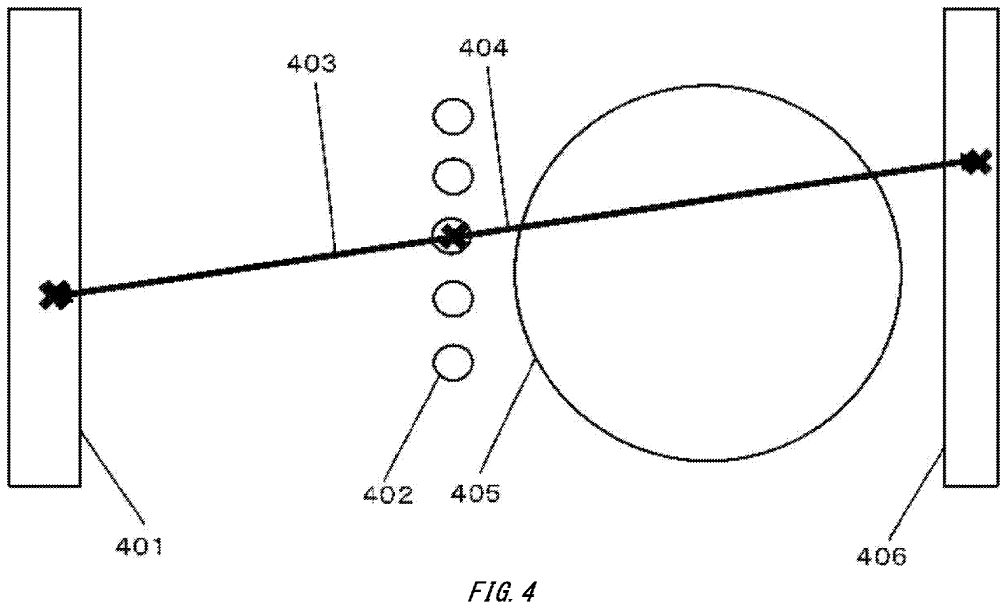

FIG. 4 is a schematic diagram of a CT according to a fifth working example One of the γ-rays emitted (radiated) in two directions opposite to each other by 180° from a radiation source 402 is made incident on a scintillator 401 on the opposite side of the measurement target to emit light. The emitted light is incident on several of a plurality of wavelength conversion fibers (not shown) disposed in two directions substantially orthogonal to the front surface and back surface of the scintillator 401, and is re-emitted in the wavelength conversion fibers. The re-emitted light is detected by a light-receiving element (not shown) attached to each end of the wavelength conversion fibers. On the other hand, another γ-ray 404 emitted from the radiation source 402 passes through a living body 405, is made incident on a scintillator 406, and emits light in the scintillator. The emitted light is incident on several wavelength conversion fibers (not shown) disposed in two directions substantially orthogonal to the front surface and the back surface of the scintillator 406, and is re-emitted in the wavelength conversion fibers. The re-emitted light is detected by a light-receiving element (not shown) attached to each end of the wavelength conversion fibers. From the information detected by these light-receiving elements, the incident position of a γ-ray 403 on the scintillator 401 and the incident position of a γ-ray 404 on the scintillator 406 can be specified. From these incident positions, the position of the radiation source 402 that emitted the γ-rays can be specified. By analyzing this information, the transmittance distribution of the γ-ray measurement target can be derived, and the two-dimensional density distribution of the measurement target can be measured (estimated). The radiation source 402, the scintillator 401, and the scintillator 406 make a plurality of measurements while rotating around the target to be measured, and measure the two-dimensional density distribution in the measurement target at each position. Then, by analyzing the information on a two-dimensional density distribution in the measurement target measured the plurality of times, the three-dimensional density distribution in the measurement target and information relating to its shape can be obtained. In the present working example, unlike the first working example, it is not necessary to dispose the radiation detector around the entire living body, so the cost may be lower than that of the first working example by the cost of the radiation detector. However, since a mechanism for rotating the scintillator and the like is required, there are issues of cost increase, reliability, and increased measurement time (diagnosis time) to rotate the scintillator and the like. It is noted that in the present working example, an example in which γ-rays are emitted from the radiation source has been described, even in a case where a radioactive ray other than the γ-ray, such as an X-ray, is emitted, it is possible to measure the three-dimensional shape in the measurement target using the configuration of the present working example. In addition, it is possible to measure the three-dimensional shape in the measurement target by using a radiation detection apparatus used in a conventional X-ray CT instead of using the scintillator and the wavelength conversion fibers in the radiation detection apparatus as in the present working example. However, the measurement method using the scintillator and the wavelength conversion fibers as in the present working example can realize a significant cost reduction of the CT apparatus.

### Sixth Working Example

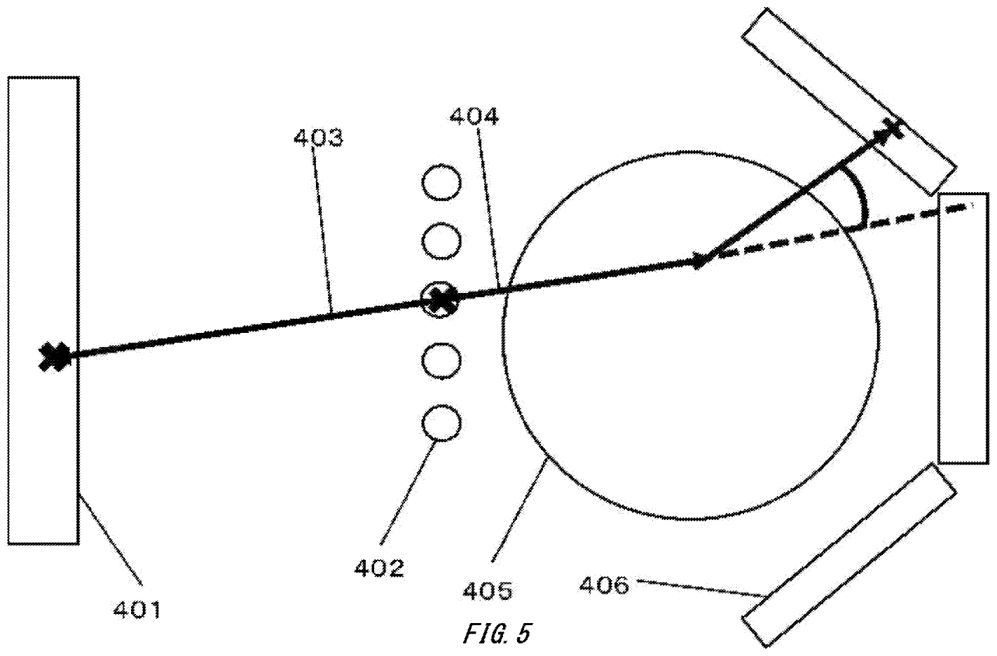

FIG. 5 is a schematic view of the sixth working example. The description of the same parts as in the fifth working example will be omitted. The present working example is different from the fifth working example in that, even when the γ-ray 404 is scattered in a living body that is the measurement target, the position of the scattered light can be determined using the method of the second working example, and the three-dimensional shape in the measurement target can be measured.

The present invention is industrially applicable as a tomography apparatus.

1. A tomography apparatus, comprising:
a γ-ray generation source configured to irradiate a measurement target with γ-rays;
a measurement unit configured to measure information relating to transmittance of a γ-ray that has passed through the measurement target; and
a substance density distribution calculation unit configured to calculate a substance density distribution of the measurement target based on the information relating to the transmittance of the γ-ray measured by the measurement unit.

2. A tomography apparatus comprising:
a γ-ray generation source configured to emit a first γ-ray in a direction of a measurement target and emit a second γ-ray to an opposite side of the measurement target;
a first scintillator configured to receive the first γ-ray emitted from the γ-ray generation source to emit light;
a plurality of first optical fibers disposed on a surface of the first scintillator and configured to re-emit light when light from the first scintillator is made incident thereon;
a first light-receiving element attached to at least one end of each of the first optical fibers and configured to measure a physical quantity relating to the light re-emitted by the first optical fibers;
a second scintillator configured to receive the second γ-ray emitted from the γ-ray generation source to emit light;
a plurality of second optical fibers disposed on a surface of the second scintillator and configured to re-emit light when the light from the second scintillator is made incident thereon;
a second light-receiving element attached to at least one end of each of the second optical fibers and configured to measure a physical quantity relating to the light re-emitted by the second optical fibers; and
a substance density distribution calculation unit configured to calculate a substance density distribution of the measurement target based on the physical quantity relating to the light measured by the first light-receiving element and the physical quantity relating to the light measured by the second light-receiving element.

3. The tomography apparatus according to claim 2, wherein
a side surface of the first scintillator includes a first measurement element for measuring information relating to energy of the first γ-ray made incident on the first scintillator,
a side surface of the second scintillator includes a second measurement element for measuring information relating to energy of the second γ-ray made incident on the second scintillator, and
the substance density distribution calculation unit calculates the substance density distribution of the measurement target based on the physical quantity relating to the light measured by the first light-receiving element, the physical quantity relating to the light measured by the second light-receiving element, the information relating to the energy of the first γ-ray measured by the first measurement element, and the information relating to the energy of the second γ-ray measured by the second measurement element.

4. The tomography apparatus according to claim 1, wherein
the measurement target is a living body.

5. The tomography apparatus according to claim 2, wherein
the measurement target is a living body.

6. The tomography apparatus according to claim 3, wherein
the measurement target is a living body.
